SEQUENCE LISTING (2 sequences)

SEQ ID NO: 1 - protein, 12 aa, Artificial sequence
  Synthesized
SFSIIHTPILPL

SEQ ID NO: 2 - protein, 12 aa, Artificial sequence
  Synthesized
TDSILRSYDWTY

(19) United States
(12) Patent Application Publication
(10) Pub. No.: US 2024/0058371 A1
(43) Pub. Date: Feb. 22, 2024

(54) MOLECULE FOR INDUCING SPONTANEOUS CALCIFICATION OF TUMOR CELLS AND USE THEREOF

(71) Applicant: ZHEJIANG UNIVERSITY, Hangzhou, Zhejiang (CN)

(72) Inventors: Ben WANG, Hangzhou, Zhejiang (CN); Jicheng WU, Hangzhou, Zhejiang (CN); Hanhui LI, Hangzhou, Zhejiang (CN); Ning TANG, Hangzhou, Zhejiang (CN)

(21) Appl. No.: 18/269,213

(22) PCT Filed: Dec. 21, 2021
(86) PCT No.: PCT/CN2021/140028

§ 371 Date: Jun. 22, 2023

(30) Foreign Application Priority Data
Dec. 22, 2020 (CN) 202011532338.3
Dec. 22, 2020 (CN) 2020115322304.4

(51) Int. Cl. A61K 31/715; A61K 31/525; C07K 16/28; A61P 35/00

(52) U.S. Cl. CPC A61K 31/715; A61K 31/525; C07K 16/2863; A61P 35/00

(57) ABSTRACT

Molecules for inducing spontaneous calcification of tumor cells and applications thereof. The molecules for inducing spontaneous calcification of tumor cells includes at least two basic units, one of the at least two basic units is a targeting functional unit that targets a molecular fragment of a functional region in the tumor cells/tissues/microenvironments, and the other basic unit is a functional unit for inducing calcification; or molecules for inducing spontaneous calcification of tumor cells comprise at least one basic unit that is both a targeting functional unit and a calcification-inducing functional unit. After calcification of tumor cells occurs, calcium salt deposition on the cell surface will affect metabolism and other physiological processes in tumor cells, leading to apoptosis induction and the suppression of tumor growth. Additionally, after calcification, tumor cells and tissues exhibit improved contrast in clinical images, thereby facilitating early and precise diagnosis of tumor lesions.

### REFERENCE TO AN ELECTRONIC SEQUENCE LISTING

The content of the electronic sequence listing (Substitute Sequence listing.txt; Size: 805 bytes; and Date of Creation: Jul. 20, 2023) is herein incorporated by reference in its entirety.

### BACKGROUND OF THE INVENTION

### Field of the Invention

The present invention relates to a class of molecules for inducing spontaneous calcification of tumor cells selectively under physiological conditions and applications thereof.

### Description of the Related Art

Chemotherapy is one of the most common treatments for cancer patients. Although surgery is able to completely remove all visible lesions, chemotherapeutic agents must be used to eliminate invisible cancer cells. Unfortunately, because most chemotherapeutic agents function by affecting cell growth and proliferation processes, they also damage normal cells. The side effects of systemic chemotherapy used to treat cancer are often severe and lead to immune system damage that causes neuropathy and neutropenia. Calcification is an important biological process in the body. For example, bones and teeth are formed by calcification; the process is also important in pathological diseases, such as atherosclerosis and urolithiasis. Calcification has also been observed in cancer, although the mechanisms are unclear; researchers have shown that tumor calcification and regional lymph node calcification are benign prognostic factors in colorectal and lung cancers. A method to induce tumor calcification via systematic injection of calcium peroxide nanoparticles was developed by a research team at East China Normal University in 2019. There have been concerns about nanoparticle accumulation in the liver and spleen because of nanomedicines used for drug delivery; further investigation is needed regarding the clinical translational safety of this approach.

In 2016, the inventors reported a new strategy to treat tumors via targeted calcification of cancer cells through the administration of high doses of folic acid (FA) and Ca²⁺. However, each FA molecule only provides two carboxyl residues that bind Ca²⁺ in biological fluids, thereby promoting nucleation of calcium minerals. Accordingly, this approach requires Ca²⁺ levels above the physiological range, which may cause hypercalcemic crisis and therefore has limited clinical applicability. Moreover, systemic FA administration promotes tumor growth, dramatically reduces body weight, and severely decreases survival time in mice.

### BRIEF SUMMARY OF THE INVENTION

To solve the problems existing in the prior art, the present invention provides a molecule for inducing spontaneous calcification of tumor cells selectively in the physiological environment in vivo. By inducing spontaneous calcification of tumors, the molecule inhibits tumor growth; improves the therapeutic effects of radiotherapy, chemotherapy, and immunotherapy; and achieves the goals of early and accurate diagnosis via contrast imaging, as well as optimal treatment.

To achieve the purpose of the present invention, the following technical solutions are provided.

The present invention discloses a molecule for inducing spontaneous calcification of tumor cells. The molecule for inducing spontaneous calcification of tumor cells comprises at least two basic units, one of the at least two basic units is a targeting functional unit that targets a molecular fragment of a functional region in tumor cells/tissues/microenvironments, and the other basic unit is a functional unit for inducing calcification; or the molecule for inducing spontaneous calcification of tumor cells comprises at least one basic unit that is both a targeting functional unit and a calcification-inducing functional unit.

There is no particular limitation for targeting functional units in the present invention, as long as they meet the requirements for targeting certain molecular fragments in tumor cells/tissues/microenvironments.

Furthermore, the functional unit for cancer cell targeting comprises one or more of an antibody targeting a tumor cell-specific surface antigen, a ligand molecule targeting a highly expressed receptor on tumor cells, a polypeptide or cyclic peptide form of that polypeptide with specific tumor cell-targeting properties, a nucleic acid aptamer with specific tumor cell-targeting properties, a polysaccharide targeting tumor cells, or a molecule targeting the tumor-specific microenvironment.

The peptides with specific tumor cell-targeting properties in the invention are able to be in linear or cyclic form.

Furthermore, the antibodies targeting tumor cell surface-specific antigens are HER2 antibodies and/or EGFR antibodies.

Furthermore, the ligand molecule targeting a highly expressed receptor on tumor cells is FA.

Furthermore, the ligand molecule targeting a highly expressed receptor in tumor cells is urokinase-type plasminogen activator receptor (uPAR).

Furthermore, the polypeptide with specific targeting for tumor cells is able to be one or more of the SP94 polypeptide targeting liver cancer cells, the TDSILRSYDWTY polypeptide targeting lung cancer cells (as shown in SEQ ID NO:2), or the RGD peptide targeting tumor blood vessels, either as a linear molecule or in its cyclic peptide form.

Among them, the sequence of the SP94 polypeptide targeting hepatocellular carcinoma cells is SFSIIHTPILPL, as shown in SEQ ID NO:1.

If the number of free carboxyl groups comprised in the targeting polypeptide molecule is greater than 5, the targeting polypeptide molecule may satisfy both targeting and calcification functions by itself, allowing the targeting polypeptide molecule to accomplish the purpose of the invention without other functional units. However, the targeting polypeptide molecules usually comprise a limited number of free carboxyl groups (n<5); it is generally difficult to simultaneously display both targeting and calcification functions.

The targeting portion of the polypeptide molecule is responsible for binding to highly expressed membrane proteins on the surface of cancer cells and preventing off-target effects. Because of the limited space available for the invention, an exhaustive list cannot be provided; however, other targeting molecules not described in the present invention also perform the same function. The calcification functional end is able to be a repetitive unit of polyglutamic acid comprising free carboxyl groups (Eₙ, n≥5), or a repetitive sequence of a casein phosphopeptide such as pS-pS-pS-pS. The calcifying region is responsible for enriching calcium and phosphate ions in the microenvironment to produce calcification. Other molecules comprising a large number of free carboxyl groups that are not extensively described in the present invention also perform the same function.

Furthermore, the tumor cell-targeting polysaccharide is hyaluronic acid, which targets CD44 on the cell surface, and/or fucoidan, which targets P-selectin on the cell surface.

Furthermore, the molecule targeting the tumor-specific microenvironment is a matrix metalloproteinase (MMP)-responsive cleaved polypeptide-hydrophobic hydrocarbon chain-hydrophilic chain and/or an alkaline phosphatase-responsive cleaved phosphate-hydrophobic hydrocarbon chain-hydrophilic chain.

Furthermore, the calcification-inducing functional unit comprises one or more strongly negatively charged groups.

The calcification-inducing functional unit satisfies the requirement of retaining a large number of strongly negatively charged groups, but other substitutions do not influence the calcification effect.

Furthermore, the strongly negatively charged groups are able to be one or more carboxyl, sulfonic acid, guanidinium, or phosphate groups.

Furthermore, the calcification-inducing functional unit is a repeating arrangement of the same functional group comprising one or more strongly negatively charged groups or a combination of different functional groups comprising one or more strongly negatively charged groups.

The number of functional groups in a molecule vary as needed; the number of monomers in a polymer macromolecule range from 1 to infinity.

Furthermore, the calcification-inducing functional unit is polysialic acid and/or polyglutamic acid.

In the invention, the molecule for inducing spontaneous calcification of tumor cells is able to be any combination of the above-mentioned targeting functional unit and calcification-inducing functional unit.

Furthermore, the molecule is able to be a folate-polysialic acid-crosslinked molecule, in which the targeting portion is able to be folate acid that is responsible for binding highly expressed receptor proteins on the surface of cancer cells to prevent off-target effects; the targeting portion is also to be any other cancer-targeting molecule. The functional end of the calcification portion is polysialic acid, which comprises a large number of free carboxyl groups and is responsible for enriching calcium and phosphate ions in the microenvironment to induce calcification.

In the present invention, the two functional regions (targeting plus inducing calcification) of the described molecules for inducing spontaneous calcification of tumor cells are both achieved by two different molecular units (targeting functional domain plus inducing calcification functional domain), such as folate-polysialic acid-crosslinked molecules; they are also combined into one a single unit with both functions (targeting plus inducing calcification). For example, hyaluronic acid is able to target CD44, a specific surface molecule on tumor stem cells, while simultaneously inducing calcification without a separate unit; fucoidan is able to target P-selectin, a tumor-specific molecule, while simultaneously inducing calcification without a separate unit.

The molecules provided by the invention for inducing spontaneous calcification of tumor cells in all types of cancers, including hematological malignancies such as leukemia, lymphoma, and multiple myeloma; gastrointestinal cancers such as esophageal cancer, gastric cancer, colorectal cancer, liver cancer, pancreatic cancer, bile duct cancer, and gallbladder cancer; respiratory system tumors such as lung cancer and pleural tumors; nervous system tumors such as glioma, neuroblastoma, and meningioma; head and neck tumors such as oral cancer, tongue cancer, laryngeal cancer, and nasopharyngeal cancer; gynecological and reproductive system tumors such as breast cancer, ovarian cancer, cervical cancer, vulvar cancer, testicular cancer, prostate cancer, and penile cancer; urological system tumors such as renal cancer and bladder cancer; and tumors of the skin and other systems such as skin cancer, melanoma, osteosarcoma, liposarcoma, and thyroid cancer.

The invention also provides the use of the above-mentioned molecules for inducing spontaneous calcification of tumor cells in the preparation of oncology drugs.

Additionally, the invention provides an oncology drug comprising the molecules for inducing spontaneous calcification of tumor cells described above.

The oncology drug comprises the molecules of the invention for inducing spontaneous calcification of tumor cells, as well as the necessary excipients; other therapeutic agents may also be included.

Furthermore, the above-mentioned drug comprising a calcification-inducing molecule is able to administered by oral, intravenous, intratumoral, or lymph node routes.

Compared with the existing technology, the present invention has the following beneficial effects:

The invention provides molecules that target tumor cells to induce the selective occurrence of spontaneous calcification; such molecules generally consist of two functional regions, a tumor-targeting region and a calcification-inducing region. The basic principle is that the targeting region of the molecule determines the cell type on which the molecule acts, and the functional region induces calcification in the physiological environment. After tumor cell calcification occurs, calcium salt deposition on the cell surface will affect metabolism and other physiological processes in tumor cells, leading to apoptosis induction and the suppression of tumor growth. Moreover, after calcification, tumor cells and tissues exhibit improved contrast in clinical images, thereby facilitating early and precise diagnosis of tumor lesions.

The molecule provided by the invention for inducing spontaneous calcification of tumor cells achieves the effects of inhibiting tumor growth, reversing resistance to other tumor chemotherapies, synergistically improving tumor radiotherapy and tumor immunotherapy, and displaying efficacy similar to vaccines. Moreover, when administered by various routes (e.g., oral, intravenous, intratumoral, and/or lymph node), the molecule changes (enhance or diminish) the results of clinical imaging such as computed tomography (CT), ultrasound, positron emission computed tomography (PET-CT), and/or magnetic resonance imaging (MRI); these changes allow earlier lesion detection or facilitate discrimination between benign and malignant tumor lesions.

The molecule comprises two functional regions that is observed through animal experiments in the present invention. The first region targets receptors on the surface of cancer cells; it selectively targets cancer cell membranes without affecting normal cells. The second region induces calcification through selective deposition of calcium and phosphate ions in the cancer cell membrane microenvironment; the resulting calcification inhibits cancer cell growth and metastasis, significantly prolonging survival as demonstrated in tumor-bearing mice. Additionally, the calcification of tumor cells and cancer tissues improves contrast in clinical images, facilitating earlier and more accurate detection and identification of microscopic tumor lesions.

In conclusion, the molecule disclosed in the present invention contribute to cancer treatment by selectively inducing tumor cell calcification; inhibiting tumor growth; reversing tumor drug resistance; improving the effects of tumor radiotherapy, chemotherapy, and immunotherapy; and prolonging the survival of patients with tumors. The calcification of tumor cells and tissues also improves contrast in clinical images, allowing earlier and more accurate detection and identification of microscopic tumor lesions.

### BRIEF DESCRIPTION OF THE DRAWINGS

FIG. 1 shows flow cytometry detection of the targeting peptide bound to various cell surfaces, as described in Example 1. The rightward shift of the peak indicates that the targeting peptide molecules selectively bound to the surface of lung cancer cells, but they did not bind to normal epithelial cells.

FIG. 2 shows the elemental spectra of lung cancer cells and normal lung epithelial cells after treatment with the calcification-inducing peptide (CiP), as determined by scanning electron microscopy-energy dispersive X-ray spectroscopy in Example 1. Compared with normal lung epithelial cells, the peaks of calcium and phosphorus elements on the surface of CiP-treated lung cancer cells are significantly increased, indicating that the targeting peptide molecules selectively induce calcification of lung cancer cells without significantly affecting normal lung epithelial cells.

FIG. 3 shows changes in the proliferative activities of various lung cancer cells and normal lung epithelial cells after treatment with different concentrations of targeting peptide, as described in Example 1. Targeting peptide-induced calcification selectively inhibited the proliferative activities of various lung cancer cells without significant toxic effects on normal lung epithelial cells.

FIG. 4 shows ultrasound imaging changes in pulmonary nodules of mice with lung cancer before and after tail vein injection of targeting peptide, as described in Example 1. The targeting peptide-induced calcification of non-small cell lung cancer is evident as high-density calcified foci on ultrasound, but the boundaries of conventional lung cancer and pulmonary nodules are unclear. The calcified lung cancer could be clearly distinguished from surrounding tissues, indicating that calcification is able to facilitate early ultrasound imaging of lung cancer and differential diagnosis of pulmonary nodules.

FIG. 5 shows CT imaging changes in pulmonary nodules of mice with lung cancer before and after tail vein injection of targeting peptide, as described in Example 1. Targeting peptide-induced calcification of non-small cell lung cancer revealed a clear boundary on CT and could be clearly distinguished from surrounding tissues, indicating that calcification is able to facilitate early CT imaging of lung cancer and differential diagnosis of pulmonary nodules.

FIG. 6 shows imaging changes in mice with lung cancer after tail vein injection of targeting peptide and calcification induction, assessed using a small animal imaging system, as described in Example 1. Targeting peptide-induced calcification of non-small cell lung cancer under physiological concentrations of calcium and phosphate ions effectively inhibited the growth and metastasis of lung cancer.

FIG. 7 shows scanning electron microscopy images of HeLa ovarian cancer cells and Ect1/E6E7 normal ovarian epithelial cells after treatment with Folate-polySia molecules, as described in Example 2. Compared with normal ovarian epithelial cells, a substantial layer of calcification was present on the surface of HeLa cells treated with Folate-polySia molecules, whereas Ect1/E6E7 cells displayed a smooth surface. These findings indicated that Folate-polySia molecules selectively induce calcification of ovarian cancer cells without significant effects on normal ovarian epithelial cells.

FIG. 8 shows changes in the proliferative activities of HeLa ovarian cancer cells and normal ovarian epithelial cells (Ect1/E6E7) after treatment with various concentrations of Folate-polySia molecules, as described in Example 2. Folate-polySia molecule-induced calcification selectively inhibited the proliferative activity of HeLa cells without significant toxic effects on normal ovarian epithelial cells.

FIG. 9 shows changes in tumor volume in a mouse subcutaneous HeLa transplantation tumor model after intraperitoneal injection of Folate-polySia molecules, as described in Example 2. Folate-polySia molecule-induced calcification of HeLa cells under physiological concentrations of calcium and phosphate ions effectively inhibited the growth of ovarian cancer cells in vivo.

FIG. 10 shows changes in survival time in a mouse subcutaneous HeLa transplantation tumor model after intraperitoneal injection of Folate-polySia molecules, as described in Example 2. Folate-polySia molecule-induced calcification of HeLa cells under physiological concentrations of calcium and phosphate ions effectively prolonged the survival time of ovarian tumor-bearing mice.

FIG. 11 shows CT scanning images of tumor calcification in a mouse subcutaneous HeLa transplantation tumor model after intraperitoneal injection of Folate-polySia-conjugated molecules, as described in Example 2. Intraperitoneal injection of Folate-polySia-conjugated molecules under physiological concentrations of calcium and phosphate ions caused tumors to exhibit substantial amounts of white calcified masses on micro-CT.

FIG. 12 shows changes in the proliferative activity of drug-resistant HeLa cells after 48 hours of treatment with various concentrations of Folate-polySia molecules, as described in Example 2. Folate-polySia molecule-induced calcification effectively inhibited the proliferative activity of drug-resistant HeLa cells, indicating that Folate-polySia molecule-induced calcification is able to kill drug-resistant HeLa cells.

FIG. 13 shows changes in the proliferative activity of drug-resistant HeLa cells after 72 hours of treatment with various concentrations of Folate-polySia molecules, as described in Example 2. Folate-polySia molecule-induced calcification for 72 hours produced a stronger killing effect in drug-resistant HeLa cells, compared with calcification for 48 hours.

FIG. 14 shows changes in cell death and survival of cisplatin-resistant HeLa cells treated with various concentrations of Folate-polySia and various concentrations of cisplatin for 48 hours, as described in Example 2. Folate-polySia treatment significantly enhanced the killing effect of cisplatin in cisplatin-resistant HeLa cells.

FIG. 15 shows changes in tumor volume in a mouse subcutaneous drug-resistant HeLa transplantation tumor model after intraperitoneal injection of Folate-polySia-conjugated molecules, as described in Example 2. Under physiological concentrations of calcium and phosphate ions, Folate-polySia molecule-induced calcification of HeLa cells effectively inhibited the growth of drug-resistant ovarian cancer cells in vivo.

FIG. 16 shows changes in survival time in a mouse subcutaneous drug-resistant HeLa transplantation tumor model after intraperitoneal injection of Folate-polySia-conjugated molecules, as described in Example 2. Under physiological concentrations of calcium and phosphate ions, Folate-polySia molecule-induced calcification of HeLa cells effectively prolonged the survival time of drug-resistant ovarian tumor-bearing mice.

FIG. 17 shows CT scanning images of tumor calcification in a mouse subcutaneous drug-resistant HeLa transplantation tumor model after intraperitoneal injection of Folate-polySia-conjugated molecules, as described in Example 2. Intraperitoneal injection of Folate-polySia-conjugated molecules under physiological concentrations of calcium and phosphate ions caused tumors to exhibit substantial amounts of white calcified masses on micro-CT, indicating that Folate-polySia induces significant calcification of drug-resistant ovarian cancer cells in vivo.

FIG. 18 shows changes in the proliferative activities of pancreatic cancer cells (KPC, Panc02), human hepatoma cells (Hep1-6), and normal human pancreatic duct epithelial (HPDE) cells treated with various concentrations of fucoidan, as described in Example 3. Fucoidan-induced calcification selectively inhibited the proliferative activity of pancreatic cancer and hepatoma cells without significant toxic effects on normal human pancreatic duct epithelial cells.

### DETAILED DESCRIPTION OF THE INVENTION

### EXAMPLE 1

Non-small cell lung cancer-targeting peptide (EₓTDSILRSYDWTY, where x is an arbitrary integer) helps to achieve early diagnosis and differential diagnosis of lung cancer by inhibiting the proliferation and metastasis of lung cancer via calcification.

The following experiments in this example were conducted using the peptide molecular sequence E₂₄ TDSILRSYDWTY, which is not specifically illustrated.

The targeting peptide is selectively bound to the surface of non-small cell lung cancer cells under physiological conditions.

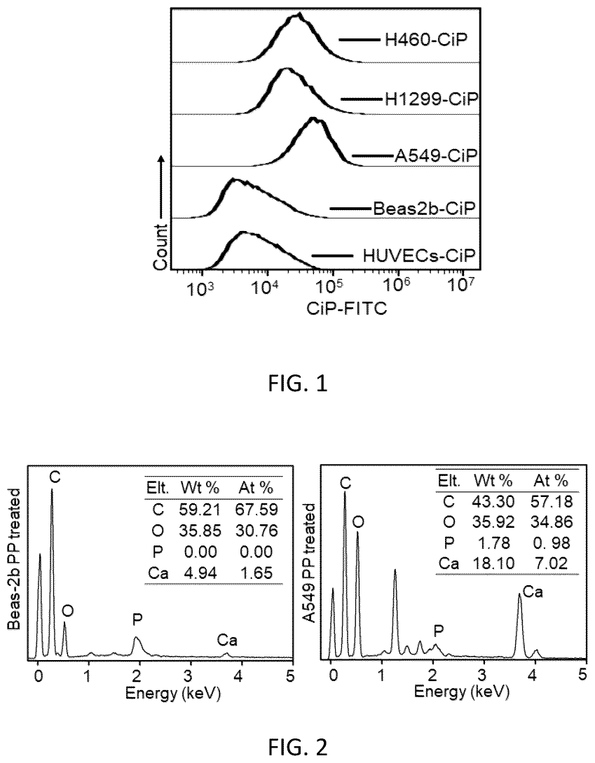

The free amino groups of aspartic acid residues in the targeting peptide were reacted with fluorescein isothiocyanate (FITC) in phosphate-buffered saline (PBS) solution for 12 hours, then dialyzed in dialysis bags for 5 days and lyophilized to obtain FITC-modified targeting peptide molecules. Under Ca²⁺ concentrations up to 2.75 mM and phosphate concentrations up to 1.61 mM, human non-small cell lung cancer cells (lines A549, H460, and H1299), Beas-2b human lung epithelial cells, and human umbilical vein endothelial cells (HUVECs) were cultured in vitro with 100 μg/mL of the above FITC-modified targeting peptide for 30 min, washed three times with PBS, and subjected to measurement of cell surface-bound targeting peptide via flow cytometry. As shown in FIG. 1, the targeting peptide selectively bound to the surface of lung cancer cells under in vitro physiological conditions; the targeting peptide weakly bound to normal cells.

The targeting peptide induces selective calcification of non-small cell lung cancer cells under physiological conditions.

A549 human non-small cell lung cancer cells were cultured in vitro for 48 hours in the presence of 1 mg/mL concentrations of the targeting peptide under Ca²⁻ concentrations up to 2.75 mM and phosphate concentrations up to 1.61 mM, then fixed with 2.5% glutaraldehyde for 48 hours, dehydrated, and dried. Subsequently, they were subjected to electron microscopy to detect calcification on the cell surface. Energy-dispersive X-ray spectroscopy (EDX) elemental scans revealed crystalline calcifications on the cell membrane after lung cancer cells had been treated with the targeting peptide, with significantly increased calcification peaks; less deposition of calcium and phosphorus elements was detected on the surface of lung epithelial cells (FIG. 2). These findings indicated that the targeting peptide is able to induce selective calcification of non-small cell lung cancer cells under physiological conditions.

Targeting peptide-induced calcification selectively kills lung cancer cells and inhibit cancer cell proliferation.

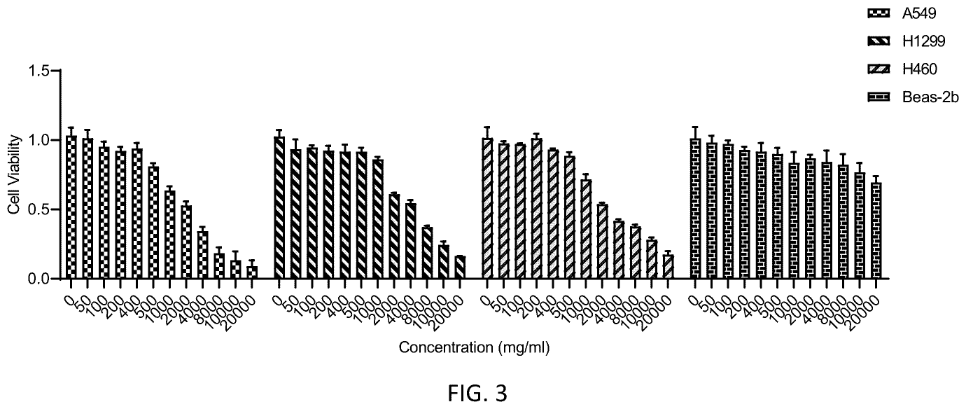

Under Ca²⁺ concentrations up to 2.75 mM and phosphate concentrations up to 1.61 mM, human non-small cell lung cancer cells (lines A549, H460, and H1299), and Beas-2b human lung epithelial cells were cultured in vitro for 48 hours with various concentrations of targeting peptide (0, 0.05, 0.1, 0.2, 0.4, 0.5, 1.0, 2.0, 4.0, 8.0, 10, and 20 mg/kg). CCK8 assays were performed to determine the killing effect of targeting peptide-induced calcification of lung cancer cells. As shown in FIG. 3, the targeting peptide-induced calcification of lung cancer cells selectively inhibited the proliferative activities of various lung cancer cells without significant toxic effects on normal lung epithelial cells.

Targeting peptide-induced calcification of non-small cell lung cancer is able to facilitate early imaging of lung cancer and differential diagnosis of pulmonary nodules.

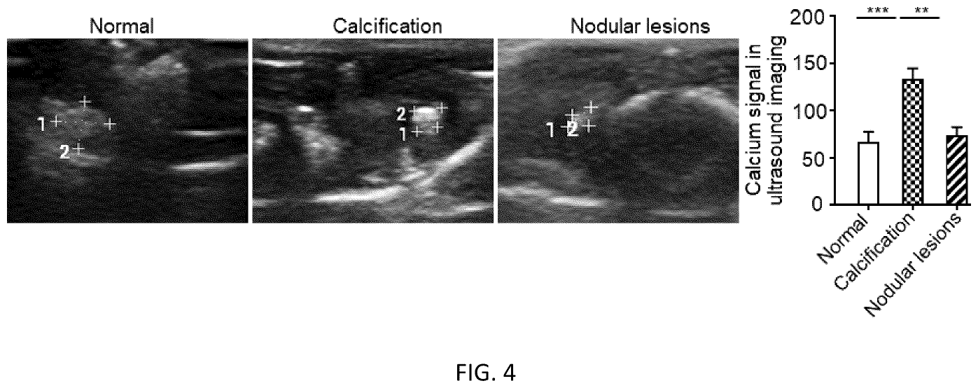

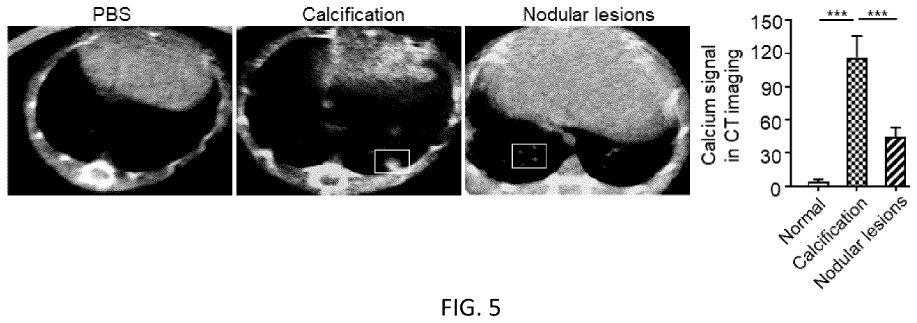

Fifty micrograms of SodA peptide (AAAIAGAFGSFDKFR) and 0.25 mL of incomplete Freund's adjuvant were mixed and subcutaneously injected into the backs of C57 mice. Two weeks later, 50 μg of SodA peptide and 6000 agarose 4B beads dissolved in 0.2 mL PBS were covalently coupled and injected into the tail vein of the same C57 mice to construct a mouse model of pulmonary nodules. Additionally, 3×10⁶ A549-Luci cells were injected into the tail vein of nude mice to construct a mouse model of pulmonary metastatic tumors. Some mice underwent intravenous injection of the targeting peptide (200 mg/kg) to induce calcification; simultaneous ultrasound and CT scans of normal lung cancer, calcified lung cancer, and pulmonary nodules were performed at 4 weeks after model establishment. The imaging results (FIG. 4) showed that targeting peptide-induced calcification of lung cancer could be clearly identified on ultrasound. After calcification, early-stage lung cancer could be visualized on CT, but conventional lung cancer could not (FIG. 5). Pulmonary nodules could not be clearly identified by imaging because of insufficient calcification. These findings indicated that targeting peptide-induced calcification of non-small cell lung cancer is able to facilitate early CT imaging of lung cancer and differential diagnosis of pulmonary nodules, thereby enhancing the sensitivity of CT-based lung cancer diagnosis.

Targeting peptide-induced calcification of non-small cell lung cancer under physiological concentrations of calcium and phosphate ions effectively inhibits the growth and metastasis of lung cancer cells.

To construct a mouse model of metastatic lung tumors, 3×10⁶ A549-Luci cells

were injected into the tail vein of nude mice, followed by injection of targeting peptide (CiP; 200 mg/kg) through the tail vein. Control mice were injected with PBS, 4 mg/kg adriamycin (Dox), and a control peptide (TDSILRSYDWTY, targeting peptide, TP) at the same concentration as the CiP peptide; this experiment investigated whether CiP targeting peptide-induced calcification of A549 cells could inhibit lung cancer growth and metastasis. Tumorigenesis was monitored using a small animal in vivo imaging system. As shown in FIG. 6, tail vein injection of the CiP targeting peptide significantly inhibited the growth of A549 cells, compared with the control group.

The above experiments were repeated using the E₆TDSILRSYDWTY, E₁₀TDSILRSYDWTY, E₁₆TDSILRSYDWTY, and E₃₀TDSILRSYDWTY peptides. All experiments showed that tail vein injection of the CiP targeting peptide significantly inhibited the growth of A549 cells. Thus, EₓTDSILRSYDWTY peptides, where x>5, significantly inhibits the growth of A549 cells.

### EXAMPLE 2

Folate-polySia molecules selectively induce spontaneous calcification of tumor cells under physiological conditions, indicating potential applications in cancer diagnosis and treatment.

Structure of an FA receptor-targeted polysialic acid-conjugated molecular drug.

Folate-polySia conjugated molecular drugs were prepared as follows: 2 mmol of FA was dissolved in 20 mL of dry dimethyl sulfoxide (DMSO) with 1 mL of redistilled triethylamine to aid solubilization; 4 mmol of N,N′-carbonyldiimidazole was added, and the mixture was stirred at room temperature for 1 hour. The reaction of N,N′-carbonyldiimidazole with FA was monitored by thin-layer chromatography with dichloromethane:methanol=3:1 as the developing solvent; ethyl acetate was used to extract the reaction solution, iodine cylinder was used for color development, and the Retention Factor Value (Rf_{CDI}) was 0.8. Four millimolar N-(aminoethyl)carbamic acid tert-butyl ester (EDA-Boc) was dissolved in 1 mL of redistilled dichloromethane, then added dropwise into the above reaction solution. The reaction was allowed to proceed overnight with stirring at room temperature. Thin-layer chromatography was used to monitor the reaction of EDA-Boc; the Rf_{Boc-NH2} was 0.5. The reaction solution was added dropwise into ether to form a precipitate, washed three times with ethyl acetate, and dried using an oil pump. The resulting solid powder was ground and dissolved in 8.8 mL of dichloromethane, then bubbled with 8.8 mL of trifluoroacetic acid. Two hours later, the reaction of folate-(EDA-Boc)₂ was detected by thin-layer chromatography with methanol as the developing solvent; the Rf value was 5/6. Dichloromethane and trifluoroacetic acid were removed by rotary evaporation; the oily product was added dropwise into diethyl ether, ground, washed three times with diethyl ether, and evaporated using an oil pump to obtain the product (N-(2-aminoethyl))₂ folate (folate-(EDA)₂). Polysialic acid (1.6 mmol) was added to 10 mL DMSO with 1 mL triethylamine to aid solubilization; 2 mmol N,N′-carbonyldiimidazole was added, and the reaction was allowed to proceed at room temperature for 24 hours. Subsequently, the reaction was quenched by dropwise addition of 0.5 mL of distilled water. The above-synthesized folate-(NH₂)₂ (1.6 mmol) was added; the reaction was conducted at room temperature for 24 hours, precipitated in ethanol, dialyzed, and lyophilized to obtain Folate-polySia complex molecules. The molecular formula of the complex molecule is shown in the figure; the complex molecule comprises an FA receptor-targeting unit and a calcification-inducing functional unit comprising multiple repeating polysialic acid monomers.

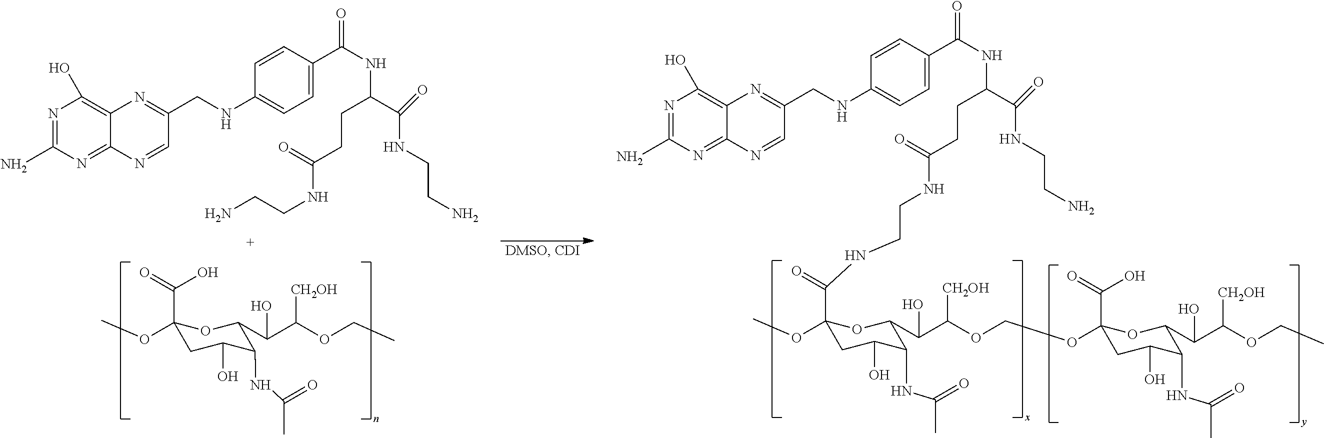

Folate-polySia-conjugated molecular drugs selectively induce calcification of folate receptor high-expressing cervical cancer cell lines.

The folate receptor high-expressing HeLa human cervical cancer cells and normal cervical epithelial Ect1/E6E7 cells were cultured in vitro with 1 mg/mL of Folate-polySia under Ca²⁺ concentrations up to 2.75 mM and phosphate concentrations up to 1.61 mM for 48 hours. As shown in FIG. 7, scanning electron microscopy revealed obvious crystalline calcification on the surface of HeLa cells, whereas no calcification was present on normal cervical epithelial cells, indicating that polysialic acid-based antitumor drugs induce selective calcification of FA receptor high-expressing cervical cancer cell lines.

Folate-polySia-conjugated molecule-induced calcification under physiological conditions selectively kills cervical cancer cells in vitro.

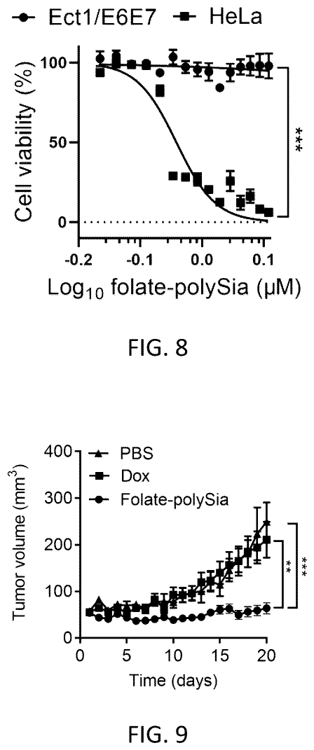

Under Ca²⁺ concentrations up to 2.75 mM and phosphate concentrations up to 1.61 mM, FA receptor high-expressing HeLa human cervical cancer cells and Ect1/E6E7 normal cervical epithelial cells were cultured in vitro for 48 hours with various concentrations of Folate-polySia-conjugated molecules (0, 0.001, 0.01, 0.02, 0.05, 0.1, 0.2, 0.5, and 1 μM). CCK8 assays were performed to determine the killing effect of Folate-polySia molecules on cancer cells. As shown in FIG. 8, Folate-polySia-induced calcification selectively killed cervical cancer cells without significant effects on Ect1/E6E7 normal cervical epithelial cells.

Under physiological conditions, Folate-polySia-conjugated molecular drugs inhibit the in vivo proliferation of FA receptor high-expressing cervical cancer cells via calcification.

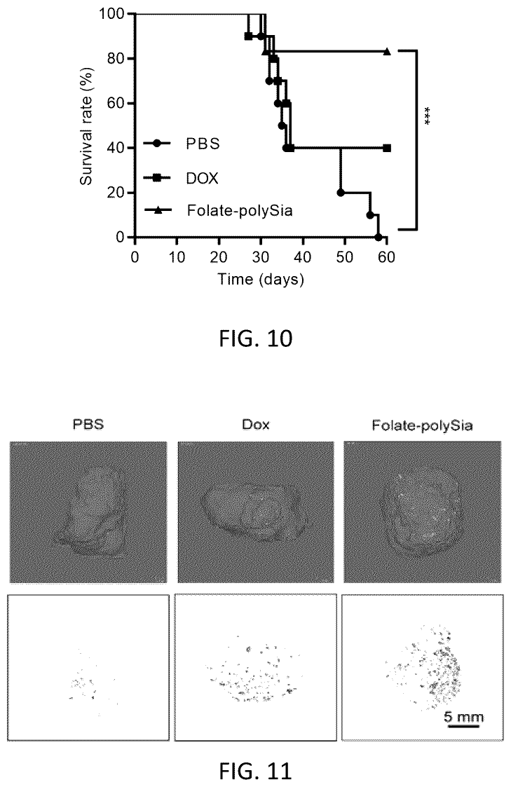

HeLa cells (5×10⁶) were subcutaneously injected into nude mice to construct a mouse subcutaneous folate receptor high-expressing HeLa transplantation tumor model; 16.7 μmol/kg of Folate-polySia were administered intraperitoneally to each mouse. In contrast, control groups were injected with PBS, as well as an equimolar concentration of folate (FA) and 5 mg/kg of adriamycin (Dox), to determine whether Folate-polySia-conjugated molecular drug-induced cervical cancer calcification could inhibit the growth of transplanted tumors in nude mice. As shown in FIG. 9, intraperitoneal injection of Folate-polySia-conjugated molecule drugs significantly inhibited the growth of transplanted tumors and significantly prolonged the survival time of nude mice (FIG. 10). Micro-CT calcium scanning of mouse tumors revealed the presence of significant calcification in the tumors of mice injected with Folate-polySia-conjugated molecule drugs (FIG. 11).

Under physiological conditions, Folate-polySia-conjugated molecular drugs reverse chemoresistance in FA receptor high-expressing cervical cancer cells via calcification.

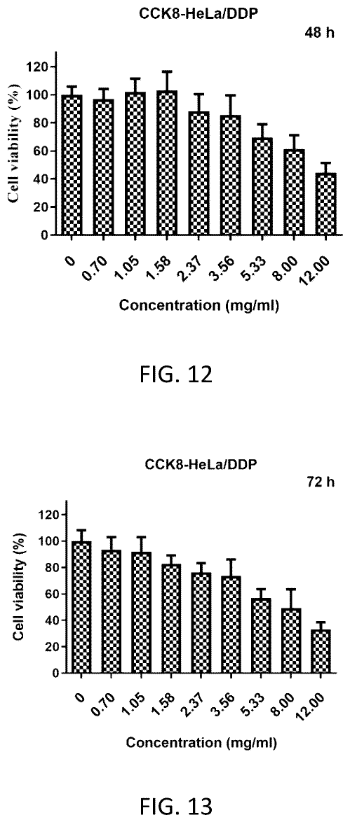

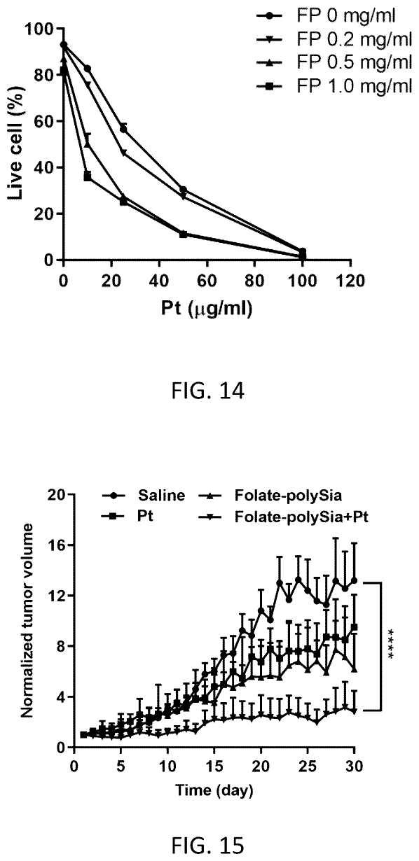

Under Ca²⁺ concentrations up to 2.75 mM and phosphate concentrations up to 1.61 mM, FA receptor high-expressing and cisplatin-resistant HeLa/DDP human cervical cancer cells were cultured in vitro with various concentrations of Folate-polySia-conjugated molecular drugs (0, 0.70, 1.05, 1.58, 2.37, 3.56, 5.33, 8.0, and 12.0 mg/mL) for 48 hours (FIG. 12) and 72 hours (FIG. 13). CCK8 assays were performed to determine the killing effects of Folate-polySia-conjugated molecular drugs on cancer cells. Appropriate concentrations of Folate-polySia-conjugated molecular drugs (0, 0.2, 0.5, and 1.0 mg/mL) were incubated in vitro for 48 hours with FA receptor high-expressing and cisplatin-resistant HeLa/DDP human cervical cancer cells under Ca²⁺ concentrations up to 2.75 mm and phosphate concentrations up to 1.61 mm, then treated with various concentrations of cisplatin (10, 25, 50, and 100 μg/mL). Live/dead staining experiments were performed to observe the killing effect of cisplatin on calcified and uncalcified cells; the results were quantified using ImageJ software (FIG. 14). As shown in FIG. 12, calcification induced by 48 hours of treatment with Folate-polySia produced a good killing effect in cisplatin-resistant HeLa cells; calcification induced by 72 hours of treatment with Folate-polySia produced a stronger killing effect in cisplatin-resistant HeLa cells (FIG. 13). Calcification induction by treatment with a low concentration of Folate-polySia for 48 hours significantly increased sensitivity to cisplatin, a chemotherapeutic agent, in cisplatin-resistant cell lines (FIG. 14). Folate-polySia-induced calcification significantly reversed the growth of cisplatin-resistant subcutaneous transplanted tumors in nude mice.

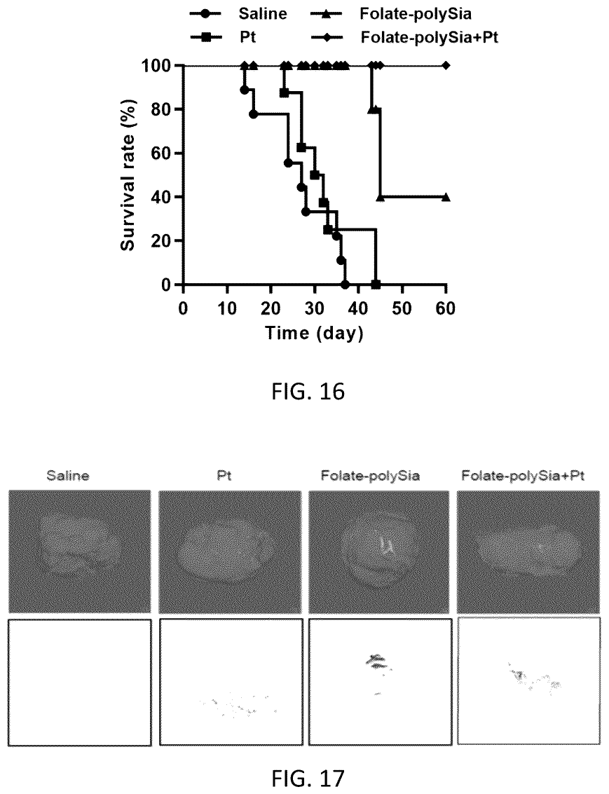

HeLa cells (5×10⁶) were subcutaneously injected into nude mice to construct a subcutaneous folate receptor high-expressing HeLa transplantation tumor model; each nude mouse underwent daily intraperitoneal administration of 16.7 μmol/kg of Folate-polySia. Mice then underwent intraperitoneal administration of physiological saline (Saline), 6.7 μmol/kg cisplatin every 5 days, or 6.7 μmol/kg cisplatin every 5 days plus 16.7 μmol/kg Folate-polySia daily to determine whether Folate-polySia-induced calcification of human cervical cancer could increase the cisplatin sensitivity of cisplatin-resistant subcutaneous transplanted tumors in nude mice. As shown in FIG. 15, intraperitoneal injection of a specific concentration of Folate-polySia significantly inhibited the subcutaneous tumor growth of HeLa/DDP cells; a small concentration of Folate-polySia plus cisplatin produced a greater inhibitory effect, indicating that Folate-polySia increased sensitivity to the chemotherapeutic agent cisplatin. Moreover, the inhibition of subcutaneous HeLa/DDP tumor growth by Folate-polySia significantly prolonged the survival time of nude mice (FIG. 16). Micro-CT calcium scanning revealed that Folate-polySia induced significant calcification of drug-resistant HeLa/DDP cells (FIG. 17).

### EXAMPLE 3

A polysaccharide drug molecule that selectively induces calcification of tumor cells under physiological conditions.

Structure of fucoidan targeting pancreatic cancer and liver cancer: molecular formula (C₆H₁₀O₇S)ₙ, where n is determined by molecular weight.

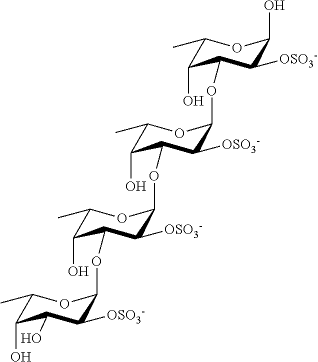

Molecular Structure of Fucoidan

Fucoidan naturally targets P-selectin molecules, which are tumor markers with high expression in pancreatic and liver cancer. This patent reveals for the first time that the fucoidan-based targeting of P-selectin molecules, highly expressed in pancreatic and liver cancer, achieves calcification and killing of cancer cells by adsorbing calcium and phosphate ions in the tumor microenvironment through the strongly negatively charged group (sulfonic acid group) in the fucoidan molecules.

Fucoidan induces calcification of pancreatic and hepatoma cells under in vitro physiological conditions.

Fucoidan (2 mg/mL) was incubated in vitro with Panc01 human pancreatic cancer cells and normal pancreatic epithelial cells under Ca²⁺ concentrations up to 2.75 mM and phosphate concentrations up to 1.61 mM for 48 hours. The cells were fixed with 2.5% glutaraldehyde for 48 hours, then subjected to dehydration and drying. Subsequently, the cells were subjected to electron microscopy to detect the calcification on the cell surface. EDX elemental scans showed that the calcification peaks on the cell membrane were significantly increased after Panc01 human pancreatic cancer cells had been treated with fucoidan; less deposition of calcium and phosphorus elements was detected on the surface of normal HPDE cells (Table 1). These findings indicated that fucoidan is able to induce selective calcification of pancreatic cancer cells under physiological conditions.

TABLE 1
Changes in the content of major elements on the cell
surface after pancreatic cancer cells and normal pancreatic
epithelial cells had been treated with fucoidan

| | | | | | |
|---|---|---|---|---|---|
| | | | | | Error |
| Elt. | Intensity | Atomic % | Atomic Ratio | Conc | 2-sig |

HPDE-fucoidan treated

| | | | | | |
|---|---|---|---|---|---|
| C | 414.950 | 60.136 | 1.653 | 53.180 | 0.875 |
| O | 110.140 | 38.664 | 1.000 | 43.991 | 1.423 |
| P | 55.410 | 0.998 | 0.030 | 2.239 | 0.138 |
| Ca | 11.670 | 0.203 | 0.006 | 0.591 | 0.093 |

Panc01-fucoidan treated

| | | | | | |
|---|---|---|---|---|---|
| C | 13.480 | 9.586 | 0.117 | 6.665 | 1.261 |
| O | 229.420 | 81.727 | 1.000 | 75.689 | 1.903 |
| P | 90.720 | 4.756 | 0.058 | 8.528 | 0.393 |
| Ca | 79.780 | 3.931 | 0.048 | 9.118 | 0.437 |

Fucoidan-induced calcification under physiological conditions in vitro selectively kills pancreatic and hepatocellular carcinoma cells.

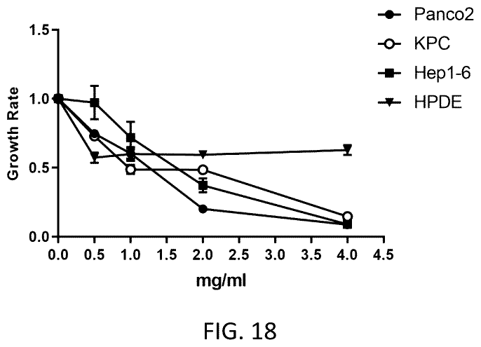

Appropriate concentrations of fucoidan molecules (0, 0.5, 1.0, 2.0, and 4.0 mg/mL) were incubated in vitro with Panc02 pancreatic cancer cells, KPC liver cancer cells, and Hep1-6 cells under Ca²⁺ concentrations up to 2.75 mM and phosphate concentrations up to 1.61 mM. The killing effects of fucoidan molecules on various cells were detected by CCK8 assays. As shown in FIG. 18, fucoidan-induced calcification produced a good killing effect in pancreatic cancer and liver cancer cells, without significant toxic effects in normal human pancreatic epithelial cells, indicating that fucoidan-induced calcification has good selective antitumor effects.

It should be understood that, after reading the above descriptions and examples regarding the invention, a person skilled in this field may make various alterations or modifications to the invention, the equivalent forms of which also fall within the scope defined by the claims appended to the present invention.

1. A molecule for inducing spontaneous calcification of tumor cells, wherein the molecule for inducing the spontaneous calcification of the tumor cells comprises at least two basic units, one of the at least two basic units is a targeting functional unit, the targeting functional unit targets a molecular region of the tumor cells or a tissue or a microenvironment, and the other basic unit is a calcification-inducing functional unit; or
wherein the molecule for inducing the spontaneous calcification of the tumor cells comprises at least one basic unit, and the at least one basic unit is both the targeting functional unit and the calcification-inducing functional unit.

2. The molecule for inducing spontaneous calcification of tumor cells according to claim 1, wherein the targeting functional unit is one or more of an antibody targeting a tumor cell surface-specific antigen, a ligand molecule targeting a highly expressed receptor on the tumor cells, a polypeptide with specific tumor cell-targeting properties or a cyclic peptide form of the polypeptide with the specific tumor cell-targeting properties, a nucleic acid aptamer with the specific tumor cell-targeting properties, a polysaccharide targeting the tumor cells, or a molecule targeting a tumor-specific microenvironment.

3. The molecule for inducing spontaneous calcification of tumor cells according to claim 2, wherein the antibody targeting the tumor cell surface-specific antigen is a human epidermal growth factor receptor 2 (HER2) antibody and/or an epidermal growth factor receptor (EGFR) antibody.

4. The molecule for inducing spontaneous calcification of tumor cells according to claim 2, wherein the ligand molecule targeting the highly expressed receptor on the tumor cells is folic acid (FA).

5. The molecule for inducing spontaneous calcification of tumor cells according to claim 2, wherein the ligand molecule targeting the highly expressed receptor on the tumor cells is urokinase-type fibrinogen activator receptor (uPAR).

6. The molecule for inducing spontaneous calcification of tumor cells according to claim 2, wherein the peptide with the specific tumor cell-targeting properties is an SP94 peptide targeting liver cancer cells, a TDSILRSYDWTY peptide targeting lung cancer cells, or an RGD (Arg-Gly-Asp) Trifluoroacetat peptide targeting tumor blood vessels.

7. The molecule for inducing spontaneous calcification of tumor cells according to claim 2, wherein the polysaccharide targeting the tumor cells is hyaluronic acid targeting CD44 on a cell surface, and/or fucoidan targeting P-selectin on the cell surface.

8. The molecule for inducing spontaneous calcification of tumor cells according to claim 2, wherein the molecule targeting the tumor-specific microenvironment is a matrix metalloproteinase (MMP)-responsive cleaved polypeptide-hydrophobic hydrocarbon chain-hydrophilic chain and/or an alkaline phosphatase-responsive cleaved phosphate-hydrophobic hydrocarbon chain-hydrophilic chain.

9. The molecule for inducing spontaneous calcification of tumor cells according to claim 1, wherein the calcification-inducing functional unit comprises one or more strongly negatively charged groups.

10. The molecule for inducing spontaneous calcification of tumor cells according to claim 9, wherein the one or more strongly negatively charged groups is one or more of a carboxyl group, a sulfonic acid group, a guanidine group, or a phosphate group.

11. The molecule for inducing spontaneous calcification of tumor cells according to claim 10, wherein the calcification-inducing functional unit is a repeated arrangement of the same functional group containing a strongly negatively charged gene or a combination of different functional groups containing a strongly negatively charged gene.

12. The molecule for inducing spontaneous calcification of tumor cells according to claim 11, wherein the calcification-inducing functional unit is polysialic acid and/or polyglutamic acid.

13. The molecule for inducing spontaneous calcification of tumor cells according to claim 12, wherein the calcification-inducing functional unit is a repeating sequence of a polyglutamic acid containing a free carboxyl group or a repeating sequence of a casein phosphopeptide.

14. The molecule for inducing spontaneous calcification of tumor cells according to claim 1, wherein the molecule for inducing the spontaneous calcification of the tumor cells is an FA-polysialic acid-conjugated molecule.

15. The molecule for inducing spontaneous calcification of tumor cells according to claim 1, wherein the molecule for inducing the spontaneous calcification of the tumor cells is hyaluronic acid and/or fucoidan.

16. The molecule for inducing spontaneous calcification of tumor cells according to claim 1, wherein the tumor cells are from leukemia, lymphoma, multiple myeloma, esophageal cancer, gastric cancer, colorectal cancer, liver cancer, pancreatic cancer, bile duct cancer, gallbladder cancer, lung cancer, pleural tumors, nervous system tumors comprising glioma, neuroblastoma, or meningioma , oral cancer, tongue cancer, laryngeal cancer, nasopharyngeal cancer, breast cancer, ovarian cancer, cervical cancer, vulvar cancer, testicular cancer, prostate cancer, penile cancer, kidney cancer, bladder cancer, skin cancer, melanoma, osteosarcoma, liposarcoma, or thyroid cancer.

17. (canceled)

18. An oncology drug, wherein the oncology drug comprises the molecule for inducing the spontaneous calcification of the tumor cells as claimed in claim 1.

19. The oncology drug according to claim 18, wherein the molecule for inducing the spontaneous calcification of the tumor cells is administered by oral, intravenous, intratumoral, or lymph node routes.

20. A cancer vaccine, wherein the cancer vaccine comprises the molecule for inducing the spontaneous calcification of the tumor cells as claimed in claim 1.

21. A method for preparing an oncology drug, comprising the following steps: preparing the oncology drug comprising the molecule for inducing the spontaneous calcification of the tumor cells as claimed in claim 1.
